(19) United States
(12) Patent Application Publication
(10) Pub. No.: US 2025/0268535 A1
(43) Pub. Date: Aug. 28, 2025

(54) SEAT AND BIOLOGICAL SENSOR

(71) Applicant: Panasonic Automotive Systems Co., Ltd., Kanagawa (JP)

(72) Inventors: Yuta NAKANO, Kanagawa (JP); Noriaki SAITO, Tokyo (JP)

(21) Appl. No.: 19/057,695

(22) Filed: Feb. 19, 2025

(30) Foreign Application Priority Data
Feb. 22, 2024 (JP) 2024-025681

(51) Int. Cl. A61B 5/00; A61B 5/0205; A61B 5/05; A61B 5/11; B60N 2/00; B60N 2/56; H05B 1/02

(52) U.S. Cl. CPC A61B 5/6893; A61B 5/0205; A61B 5/05; A61B 5/1116; B60N 2/0022; B60N 2/003; B60N 2/0035; B60N 2/5678; H05B 1/0238

(57) ABSTRACT

Provided is a seat that includes: a heater element that meanders; and a first biological sensor including a first emission region which, in operation, emits a first electromagnetic wave in a first direction. The first emission region has a dimension in a second direction, which is a direction perpendicular to the first direction, and a dimension in a third direction, which is a direction perpendicular to both the first direction and the second direction, where the dimension in the second direction is smaller than the dimension in the third direction. The heater element and the first emission region are arranged in different positions in the second direction.

### TECHNICAL FIELD

The present disclosure relates to a seat and a biological sensor.

### BACKGROUND ART

In recent years, the development of a 60 GHz radar built into a seat for use in biological sensing has been advanced. Further, with the increasing popularity of electric vehicles for which it is difficult to utilize engine waste heat, the demand for seat heaters is increasing.

Patent Literature (hereinafter referred to as “PTL”) 1 discloses a seat in which the arrangement of a heater element is devised and a sensor is arranged in the center of the seat with a member, which interferes with the passage of electromagnetic waves, on the left and right sides. Further, PTL 2 discloses a seat in which a respiration sensor is arranged at a low-temperature seat heater.

### CITATION LIST

### Patent Literature

PTL 1
Japanese Patent Application Laid-Open No. 2022-040354
PTL 2
Japanese Patent Application Laid-Open No. 2013-154854

### SUMMARY OF INVENTION

### Technical Problem

However, in the technology of PTL 1, there is a constraint on the heater arrangement, which may therefore lead to a decrease in the thermal efficiency of the heater. Further, in the technology of PTL 2, the use of a piezoelectric film sensor for biological sensing is considered, but the use of a radio wave sensor is not considered, and there is a possibility that radio waves may be shielded.

One non-limiting and exemplary embodiment facilitates providing a seat and a biological sensor each capable of suppressing a constraint on the heater arrangement and increasing the thermal efficiency of a heater.

### Solution to Problem

For this purpose, an aspect of a seat according to the present disclosure includes: a heater element that meanders; and a first biological sensor including a first emission region which, in operation, emits a first electromagnetic wave in a first direction. The first emission region has a dimension in a second direction, which is a direction perpendicular to the first direction, and a dimension in a third direction, which is a direction perpendicular to both the first direction and the second direction, where the dimension in the second direction is smaller than the dimension in the third direction. The heater element and the first emission region are arranged in different positions in the second direction.

In addition, an aspect of a biological sensor configured to be installed on a sheet which is included a heater element arranged in a meandering manner according to the present disclosure includes: signal generation circuitry which, in operation, generates a signal; and an antenna which, in operation, emits the signal as a first electromagnetic wave in a first direction through a first emission region. The first emission region has a dimension in a second direction and a dimension in a third direction, the second direction being a direction perpendicular to the first direction, the third direction being a direction perpendicular to both the first direction and the second direction, the dimension in the second direction being smaller than the dimension in the third direction, and the heater element and the first emission region are arranged in different positions in the second direction.

### Advantageous Effects of Invention

According to the present disclosure, it is possible to suppress a constraint on the heater arrangement and to increase the thermal efficiency of a heater.

### BRIEF DESCRIPTION OF DRAWINGS

FIG. 1 is a diagram illustrating an exemplary arrangement of a heater element and a biological sensor;

FIG. 2A is a diagram illustrating an exemplary arrangement of the heater element and a biological sensor;

FIG. 2B is a diagram illustrating an exemplary state in which the back of a person is away from a seat;

FIG. 3 is a cross-sectional view illustrating an exemplary arrangement of a biological sensor in which an end-fire array antenna is mounted;

FIG. 4 is a diagram illustrating an exemplary biological sensor that emits an electromagnetic wave with horizontal polarization;

FIG. 5 is a diagram illustrating the positions of a surface including meandering portions of the heater element and biological sensors;

FIG. 6A is a diagram illustrating an exemplary arrangement of biological sensors each including a side surface having a small dimension in the width direction;

FIG. 6B is a diagram illustrating an exemplary arrangement of a biological sensor including a side surface having a small dimension in the height direction and biological sensors each including a side surface having a small dimension in the width direction;

FIG. 7 is a diagram illustrating exemplary functional blocks of a biological sensor;

FIG. 8A is a diagram illustrating an exemplary configuration of a biological sensor; and

FIG. 8B is a diagram illustrating an exemplary configuration of the biological sensor.

### DESCRIPTION OF EMBODIMENTS

Hereinafter, an embodiment of the present disclosure will be described with reference to the accompanying drawings. Note that, any of the embodiment described below illustrates a specific example of the present disclosure. Accordingly, each component, the arrangement position and connection form of each component, and the like illustrated in the following embodiment are examples and are not intended to limit the present disclosure. In addition, components that are not recited in any of the independent claims among the components in the following embodiment will be described as optional components.

Each drawing is a schematic diagram and is not necessarily a precise illustration. In each drawing, the same reference signs are attached to the substantially same configurations, and redundant descriptions will be omitted or simplified.

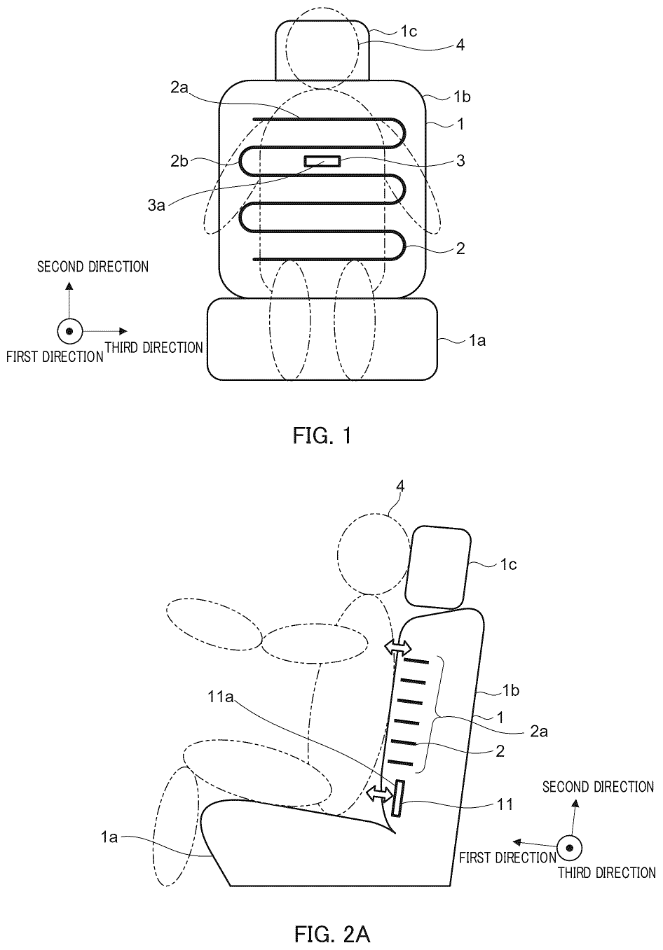

First, seat 1 to be installed in a vehicle will be described. As illustrated in FIG. 1, seat 1 includes heater element 2 and biological sensor 3.

Seat 1 includes: seat cushion 1a which supports the buttocks and thighs of person 4; seat back 1b whose lower end portion is supported by seat cushion 1a and which serves as a backrest; and headrest 1c which is provided at seat back 1b and supports the head of person 4.

Heater element 2 is arranged in a meandering manner inside seat back 1b of seat 1. In the example of FIG. 1, heater element 2 has a shape in which a plurality of straight line portions 2a and a plurality of curved line portions 2b having a U shape are alternately arranged side by side.

Biological sensor 3 is a sensor that emits electromagnetic waves from emission region 3a in a first direction and measures biological information such as skin vibrations caused by the respiration or heartbeat of person 4. Biological sensor 3 is, for example, includes an end-fire array (EFA) antenna mounted therein, which has radiation directivity of electromagnetic waves in a board edge direction, and detects displacement of the body surface of person 4 by the electromagnetic waves.

This biological sensor 3 is a thin sensor that includes emission region 3a that emits electromagnetic waves in a first direction and has a dimension in a height direction (second direction) and a dimension in a width direction (third direction) where the dimension in the height direction (second direction) is smaller than the dimension in the width direction (third direction). The dimension (thickness) of emission region 3a in the height direction is, for example, one cm. Note that, biological sensor 3 may be a sensor including an antenna mounted therein, which is other than an end-fire array antenna.

Then, when viewed from the direction (first direction) perpendicular to a surface including meandering portions of heater element 2, emission region 3a is arranged in a gap created by heater element 2 inside the meandering portions. In the example of FIG. 1, emission region 3a is arranged in a position sandwiched by heater element 2 between two straight portions 2a of heater element 2, which face each other, when viewed from the direction perpendicular to the surface including the meandering portions of heater element 2. With respect to emission region 3a, heater element 2 and emission region 3a are arranged in different positions in the second direction.

By arranging emission region 3a, which emits radio waves to the outside, in a gap created by heater element 2 in this manner, it is possible to increase the degree of freedom in the arrangement of heater element 2 without inhibiting the emission of electromagnetic waves, and to improve the thermal efficiency of a heater. Note that, the detailed internal configuration of biological sensor 3 will be described later.

Note that, in a case where a biological sensor is provided at the seat of the vehicle, the measurement of biological information may become unstable since, the distance between seat 1 and the driver greatly changes due to a vehicle body vibration when the driver drives without leaning back against seat 1. For this reason, a biological sensor may be arranged as described below.

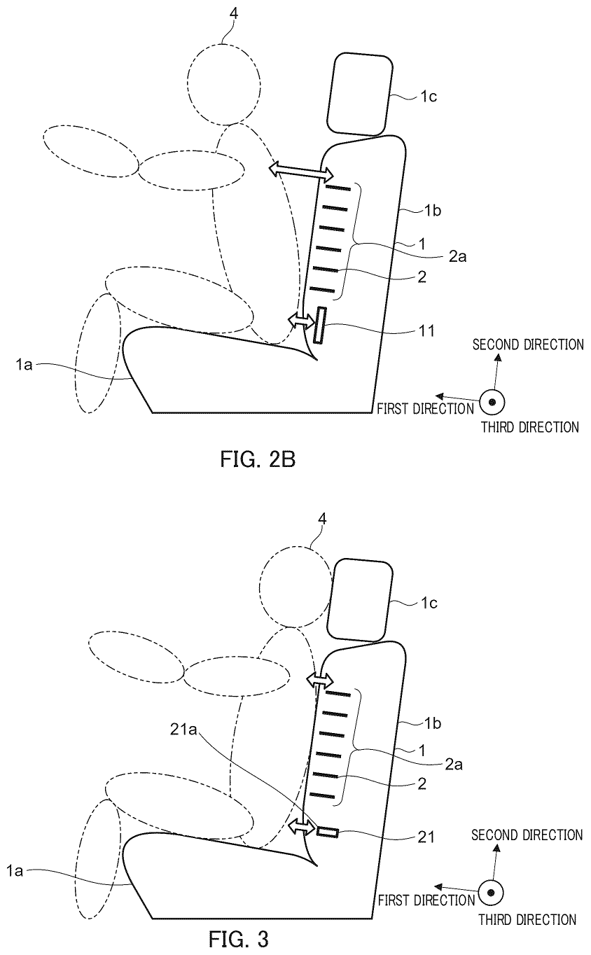

Specifically, as illustrated in FIG. 2A, biological sensor 11 is arranged in a lower region of seat back 1b below heat generation region 2a of heater element 2. Heat generation region 2a is a region in which heater element 2 is provided to warm person 4, and heat generation region 2a is, for example, a region in which heater element 2 meanders as illustrated in FIG. 1. With respect to the position in which biological sensor 11 is arranged, biological sensor 11 is arranged, in the lower region of seat back 1b, in a position closer to seat cushion 1a than heat generation region 2a of heater element 2 in the second direction. The position in which biological sensor 11 is arranged is, for example, a position within five cm to ten cm from a position in which seat cushion 1a and seat back 1b are in contact with each other.

In a case where heater element 2 is provided in both seat cushion 1a and seat back 1b, the heat generation portion of heater element 2 is often divided between seat cushion 1a and seat back 1b, and in a lower region of seat cushion 1a below heat generation region 2a, seat 1 and person 4 are not in contact with each other as illustrated in FIG. 2A. For this reason, even when biological sensor 11 is arranged in that region, the heating efficiency does not decrease.

Further, in a case where person 4 does not lean back against seat back 1b and drives in a forward-leaning posture as illustrated in FIG. 2B, the state position significantly moves due to the influence of the road surface, and the distance between person 4 and biological sensor 11 is likely to vary when biological sensor 11 is in a high position.

However, although person 4 may sit at the edge of seat cushion 1a, the position in which seat cushion 1a and the buttocks of person 4 are in contact with each other does not change significantly, and thus, the distance between biological sensor 11 and person 4 is unlikely to vary in a case where biological sensor 11 is in a low position. Thus, the measurement of biological information is stabilized.

Note that, biological sensor 11 may be a sensor in which an end-fire array antenna is mounted, or may be a sensor in which another type of antenna is mounted. Further, both biological sensor 3 illustrated in FIG. 1 and biological sensor 11 illustrated in FIGS. 2A and 2B may be provided at seat 1, or at least one thereof may be provided at seat 1.

FIG. 3 illustrates a case where biological sensor 21 in which an end-fire array antenna is mounted is provided in the lower region of seat cushion 1a, which is below heat generation region 2a of seat cushion 1a. Here, biological sensor 21 includes emission region 21a for electromagnetic waves, which has a dimension in the height direction (second direction) and a dimension in the width direction (third direction) where the dimension in the height direction (second direction) is smaller than the dimension in the width direction (third direction), and biological sensor 21 emits electromagnetic waves from emission region 21a to the outside.

By using such biological sensor 21, it is possible to arrange biological sensor 21 even when heat generation region 2a of heater element 2 is arranged considerably lower in seat back 1b.

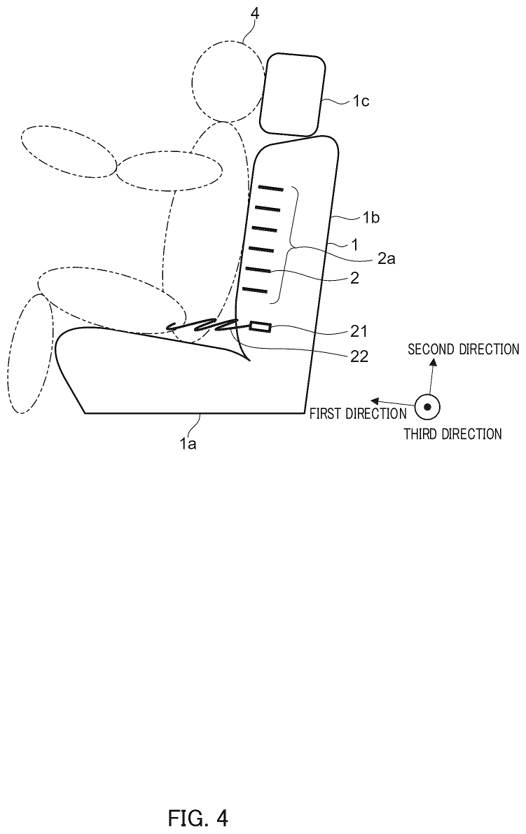

Further, as illustrated in FIG. 4, biological sensor 21 may emit electromagnetic wave 22 with horizontal polarization. Below heat generation region 2a of heater element 2, a lead wire that supplies electric power to heat generation region 2a extends substantially in the vertical direction, but it is possible to reduce the influence of the lead wire in the measurement of biological information by emitting electromagnetic wave 22 with horizontal polarization.

Note that, the emission region of the biological sensor that emits electromagnetic waves to the outside of the biological sensor may be arranged such that the emission region may be arranged not to overlap with heater element 2 on the surface including the meandering portions of heater element 2, or may be arranged forward from the surface including the meandering portions.

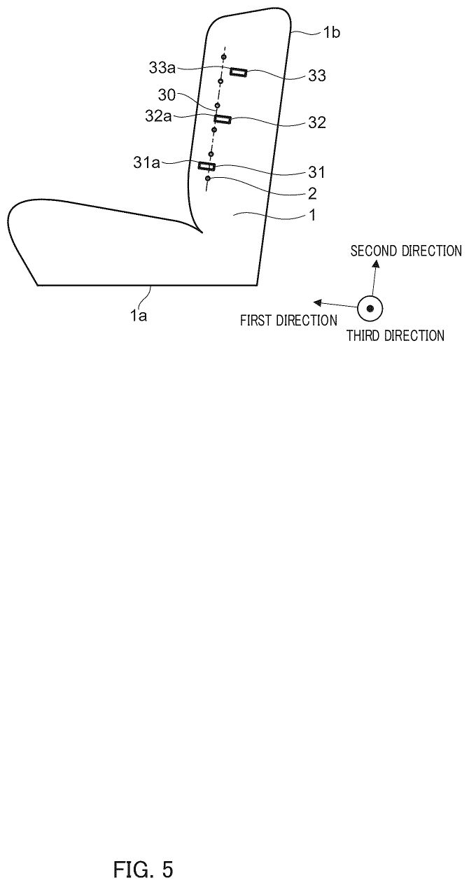

FIG. 5 illustrates surface 30 including the meandering portions of heater element 2 and biological sensors 31, 32 and 33 provided in different positions. For example, in the first direction, the emission region and heater element 2 are arranged in the identical position or the emission region is arranged in a position closer to the outer side of seat back 1b than heater element 2.

Emission region 31a of biological sensor 31 is arranged rearward from surface 30 including the meandering portions of heater element 2. In this case, when emission region 31a is arranged in a gap created by heater element 2 inside the meandering portions of heater element 2 as viewed from the direction (first direction) perpendicular to surface 30 including the meandering portions, it is possible to prevent electromagnetic waves from being affected by heater element 2.

Emission region 32a of biological sensor 32 is arranged at surface 30 including the meandering portions of heater element 2 such that emission region 32a does not overlap with heater element 2. Further, emission region 33a of biological sensor 33 is arranged forward from surface 30 including the meandering portions of heater element 2.

By arranging biological sensors 32 and 33 in this manner, it is possible to further prevent electromagnetic waves from being affected by heat element 2.

Further, although it is configured in FIG. 1 such that biological sensor 3 including emission region 3a which has a dimension in the height direction and a dimension in the width direction where the dimension in the height direction is smaller than the dimension in the width direction is arranged in a gap created by heater element 2 has been described, an emission region of a biological sensor, in which the emission region has a dimension in the width direction and a dimension in the height direction where the dimension in the width direction is smaller than the dimension in the height direction, and emits electromagnetic waves to the outside, may be arranged in a region sandwiched between two portions of one or more heater elements 2.

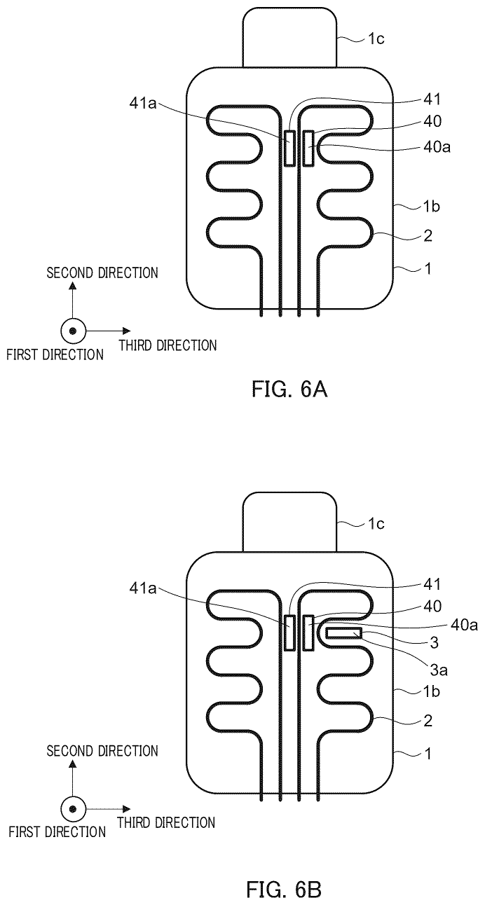

FIG. 6A illustrates biological sensors 40 and 41 including emission regions 40a and 41a, respectively, in which each of emission regions 40a and 41a has a dimension in the width direction (third direction) and a dimension in the height direction (second direction) where the dimension in the width direction (third direction) is smaller than the dimension in the height direction (second direction).

FIG. 6B illustrates biological sensor 3 including emission region 3a, which has a dimension in the height direction and a dimension in the width direction where the dimension in the height direction is smaller than the dimension in the width direction, and biological sensors 40 and 41 including emission regions 40a and 41a, respectively, in which each of emission regions 40a and 41a has a dimension in the width direction and a dimension in the height direction where the dimension in the width direction is smaller than the dimension in the height direction.

Heater element 2 includes portions that are linearly arranged in the second direction and in the third direction, respectively. Each of emission regions 40a and 41a is arranged in a position sandwiched by heater element 2 in the second direction, and emission region 3a is arranged in a position sandwiched by heater element 2 in the third direction.

Even in the case of wiring of heater element 2 in which it is difficult to arrange emission region 3a having a small dimension in the height direction in a gap, the use of biological sensors 40 and 41 which include emission regions 40a and 41a, respectively, each of which has a small dimension in the width direction, makes it possible to easily arrange biological sensors 40 and 41, and makes it possible to increase the degree of freedom in the wiring of heater element 2.

Note that, although two biological sensors 40 and 41 are illustrated together with biological sensor 3 in FIG. 6B, the biological sensor(s) to be arranged may be any one of the three biological sensors or may be two of the three biological sensors.

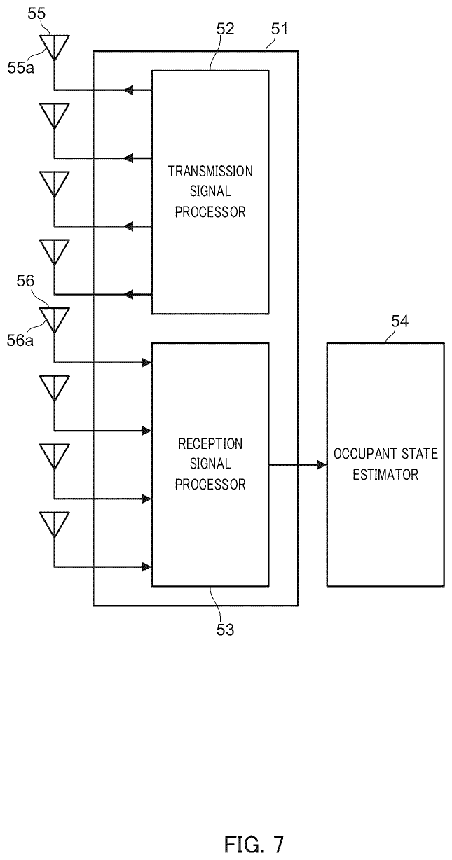

Next, exemplary functional blocks of each biological sensor illustrated in FIGS. 1 to 6 will be described. As illustrated in FIG. 7, each biological sensor includes signal processing IC 51, occupant state estimator 54, transmission antenna 55, and reception antenna 56. Further, signal processing IC 51 includes transmission signal processor 52 and reception signal processor 53.

Signal processing IC 51 constitutes, for example, a radar apparatus of a frequency modulated continuous wave (FM-CW) system. In this respect, however, a radar apparatus of a pulse radar system may also be configured.

Transmission signal processor 52 is connected to a plurality of antenna elements 55a of transmission antenna 55. Further, reception signal processor 53 is connected to a plurality of antenna elements 56a of reception antenna 56.

Transmission signal processor 52 controls the direction of an electromagnetic wave to be transmitted from a biological sensor to the outside by, for example, electronic scanning. Transmission signal processor 52 continuously generates, for example, by using a reference signal acquired from an oscillator, a transmission signal of a high frequency (for example, a millimeter wave frequency band) which has been subjected to frequency modulation processing such that the frequency increases and decreases repeatedly timewise.

Then, transmission signal processor 52 transmits the transmission signal to each antenna element 55a, causing each antenna element 55a to transmit an electromagnetic wave that has been frequency-modulated.

Note that, transmission signal processor 52 changes the direction of an electromagnetic wave (for example, a synthetic wave of electromagnetic waves to be transmitted from each antenna element 55a) to be transmitted from a biological sensor to the outside by adjusting the phase of the electromagnetic waves to be transmitted from each antenna element 55a.

Note that, transmission signal processor 52 may cause each antenna element 55a to transmit radio waves simultaneously (for example, in a frequency division multiplexing scheme or a code division multiplexing scheme) or in a time division manner (for example, in a time division multiplexing scheme). Note that, the direction of an electromagnetic wave (for example, a synthetic wave of electromagnetic waves to be transmitted from each antenna element 55a) to be transmitted from a biological sensor to the outside may be changed by adjusting each phase between antennas of the electromagnetic waves to be transmitted.

Reception signal processor 53 performs, for example, by using a local signal to be generated by transmission signal processor 52, orthogonal detection processing, frequency analysis processing, and the like on a reception signal related to a reflection wave acquired from each antenna element 56a. Further, reception signal processor 53 calculates the phase difference of a reflected wave received by each antenna element 56a, and estimates the distance and/or azimuth to the human body, the relative velocity, and the like based on the phase difference.

Occupant state estimator 54 estimates the state of person 4, such as the respiration and heartbeat of person 4, based on the information estimated by reception signal processor 53, and transmits, according to the estimation result, a control signal to a vehicle electronic control unit (ECU) to activate the brake or the like.

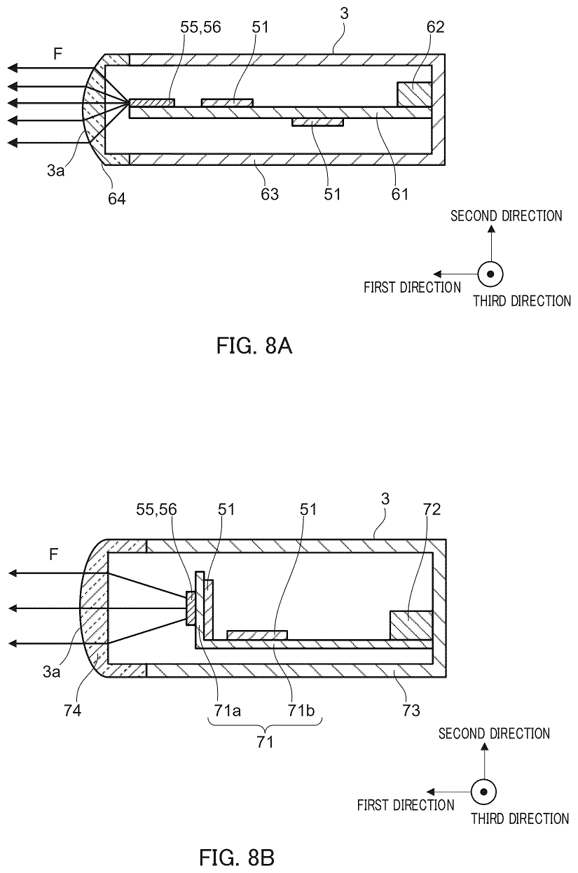

Next, an exemplary configuration of biological sensor 3 will be described. FIG. 8A illustrates biological sensor 3 including emission region 3a that emits electromagnetic waves to the outside and has a dimension in the height direction (second direction) and a dimension in the width direction (third direction) where the dimension in the height direction (second direction) is smaller than the dimension in the width direction (third direction).

Biological sensor 3 includes two signal processing ICs 51, transmission antenna 55, reception antenna 56, circuitry board 61, connector 62, housing 63, and dielectric lens 64. Further, solid line arrow F represents an electromagnetic wave transmitted by transmission antenna 55.

Circuitry board 61 is a board in which transmission antenna 55, reception antenna 56, signal processing ICs 51, connector 62, and the like are mounted.

Signal processing ICs 51, transmission antenna 55, reception antenna 56, connector 62, and the like are mounted within a board surface of the front surface or rear surface of circuitry board 61, and wirings (not illustrated) that electrically connect the respective mounted components to each other are pattern-formed. Circuitry board 61 is disposed such that the extending direction of the board surfaces thereof is parallel to the front-rear direction. Note that, the direction close to dielectric lens 64 is the front direction (front end direction) of circuitry board 61.

Transmission antenna 55 is disposed in a front region of circuitry board 61, and transmits electromagnetic waves toward the front end direction of circuitry board 61 and parallel to the board surfaces of circuitry board 61. Further, reception antenna 56 is disposed in the front region of circuitry board 61, and receives a reflected wave that is incident from the front end direction of circuitry board 61 and is caused to be parallel to the board surfaces of circuitry board 61 by dielectric lens 64. For this reason, transmission antenna 55 and reception antenna 56 have transmission and reception directivity in the front end direction of circuitry board 61. Note that, the region of circuitry board 61, where the region is close to dielectric lens 64, is the front region of circuitry board 61.

As transmission antenna 55 and reception antenna 56, typically, an end-fire array antenna having directivity in the direction of a side of the front end of circuitry board 61 is applied. Note that, the end-fire array antenna is configured to include a plurality of strip conductors arrayed such that the longitudinal directions of the plurality of strip conductors are parallel to each other, and the end-fire array antenna transmits and receives electromagnetic waves along the direction in which the plurality of strip conductors is arrayed.

Note that, transmission antenna 55 and reception antenna 56 may be constituted by a conductor pattern formed at circuitry board 61, and other than the end-fire array antenna, a Yagi array antenna, a Fermi antenna, a post wall waveguide antenna, a post wall horn antenna or the like may also be applied. Further, transmission antenna 55 and reception antenna 56 may be constituted by a single antenna that is shared in the transmission and reception of electromagnetic waves.

FIG. 8B illustrates another configuration of biological sensor 3. This biological sensor 3 includes two signal processing ICs 51, transmission antenna 55, reception antenna 56, circuitry board 71, connector 72, housing 73, and dielectric lens 74. Further, solid line arrow F represents an electromagnetic wave transmitted by transmission antenna 55.

Circuitry board 71 is a board in which signal processing ICs 51, transmission antenna 55, reception antenna 56, connector 72, and the like are mounted. Signal processing ICs 51, transmission antenna 55, reception antenna 56, connector 72, and the like are mounted within a board surface of the front surface or rear surface of circuitry board 71, and wirings (not illustrated) that electrically connect the respective mounted components to each other are pattern-formed.

Circuitry board 71 includes first board 71a, in which the board surfaces extend in the up-down direction and which is disposed such that one board surface faces forward, and second board 71b, which is disposed such that the board surfaces extend in the front-rear direction.

First board 71a is a board for disposing signal processing IC 51, transmission antenna 55, and reception antenna 56. Second board 71b is a board for disposing components, such as signal processing IC 51, connector 72, and an electrolytic capacitor (not illustrated), other than transmission antenna 55 and reception antenna 56. Note that, second board 71b and first board 71a are electrically connected to each other by a wiring (not illustrated).

Note that, the biological sensors illustrated in FIGS. 2A to 6B are not necessarily have the configuration illustrated in FIG. 8A or 8B, and may have a configuration in which the configuration illustrated in FIG. 8A or 8B is rotated by 90 degrees around an axis in the longitudinal direction, or may have another configuration.

The present disclosure also encompass other forms obtained by making various modifications conceivable to those skilled in the art to each embodiment, or forms implemented by arbitrarily combining components and functions in each embodiment without departing from the spirit of the present disclosure.

The disclosure of Japanese Patent Application No. 2024-025681, filed on Feb. 22, 2024, including the specification, drawings and abstract, is incorporated herein by reference in its entirety.

### INDUSTRIAL APPLICABILITY

The present disclosure can be utilized in a seat provided with a heat element and a biological sensor.

1. A seat, comprising:
a heater element that meanders; and
a first biological sensor including a first emission region which, in operation, emits a first electromagnetic wave in a first direction, wherein
the first emission region has a dimension in a second direction and a dimension in a third direction, and the heater element and the first emission region are arranged in different positions in the second direction, the second direction being a direction perpendicular to the first direction, the third direction being is a direction perpendicular to both the first direction and the second direction, the dimension in the second direction being smaller than the dimension in the third direction.

2. The seat according to claim 1, wherein
in the first direction, the first emission region and the heater element are arranged in an identical position or the first emission region is arranged in a position closer to an outer side of the seat than the heater element.

3. The seat according to claim 1, wherein
the first emission region is arranged in a position sandwiched by the heater element in the second direction.

4. The seat according to claim 1, wherein
the first electromagnetic wave is an electromagnetic wave with horizontal polarization.

5. The seat according to claim 1, wherein:
the seat includes a seat back and a seat cushion,
the first biological sensor and the heater element are arranged in the seat back of the seat, and
the first biological sensor is arranged in a position closer to the seat cushion than a heat generation region of the heater element in the second direction.

6. The seat according to claim 5, wherein
the position in which the first biological sensor is arranged is a position within five cm to ten cm from a position in which the seat cushion and the seat back are in contact with each other.

7. The seat according to claim 5, wherein
the first electromagnetic wave is an electromagnetic wave with horizontal polarization.

8. The seat according to claim 1, wherein:
the seat includes a seat back and a seat cushion,
the seat further includes a second biological sensor which, in operation, emits a second electromagnetic wave,
the first biological sensor, the second biological sensor, and the heater element are arranged in the seat back of the seat, and
the second biological sensor is arranged in a position closer to the seat cushion than a heat generation region of the heater element in the second direction.

9. The seat according to claim 8, wherein
the second biological sensor includes a second emission region which, in operation, emits the second electromagnetic wave in the first direction and has the dimension in the second direction and the dimension in the third direction, the dimension in the second direction being smaller than the dimension in the third direction.

10. The seat according to claim 1, further comprising a second biological sensor including a second emission region which, in operation, emits a second electromagnetic wave in the first direction and has the dimension in the third direction and the dimension in the second direction, the dimension in the third direction being smaller than the dimension in the second direction, wherein:
the heater element includes portions that are linearly arranged in the second direction and in the third direction, respectively,
the first emission region is arranged in a position sandwiched by the heater element in the second direction, and
the second emission region is arranged in a position sandwiched by the heater element in the third direction.

11. The seat according to claim 8, wherein
the second electromagnetic wave is an electromagnetic wave with horizontal polarization.

12. The seat according to claim 8, wherein
at least one of the first biological sensor and/or the second biological sensor includes an end-fire array antenna.

13. A biological sensor configured to be installed on a sheet which is included a heater element arranged in a meandering manner, comprising:
signal generation circuitry which, in operation, generates a signal; and
an antenna which, in operation, emits the signal as a first electromagnetic wave in a first direction through a first emission region, wherein:
the first emission region has a dimension in a second direction and a dimension in a third direction, the second direction being a direction perpendicular to the first direction, the third direction being a direction perpendicular to both the first direction and the second direction, the dimension in the second direction being smaller than the dimension in the third direction, and
the heater element and the first emission region are arranged in different positions in the second direction.

14. The biological sensor according to claim 13, wherein
in the first direction, the first emission region and the heater element are arranged in an identical position or the first emission region is arranged in a position closer to an outer side of the seat than the heater element.

15. The biological sensor according to claim 13, wherein
the first emission region is arranged in a position sandwiched by the heater element in the second direction.

16. The biological sensor according to claim 13, wherein
the first electromagnetic wave is an electromagnetic wave with horizontal polarization.

17. The biological sensor according to claim 13, wherein:
the seat includes a seat back and a seat cushion,
the biological sensor and the heater element are arranged in the seat back of the seat, and
the biological sensor is arranged in a position closer to the seat cushion than a heat generation region of the heater element in the second direction.

18. The biological sensor according to claim 17, wherein
the position in which the biological sensor is arranged is a position within five cm to ten cm from a position in which the seat cushion and the seat back are in contact with each other.

19. The biological sensor according to claim 17, wherein
the first electromagnetic wave is an electromagnetic wave with horizontal polarization.

20. The biological sensor according to claim 13, wherein
the antenna is configured as an end-fire array antenna.
